(19) United States
(12) Patent Application Publication
(10) Pub. No.: US 2026/0232176 A1
(43) Pub. Date: Aug. 13, 2026

(54) SYSTEMS AND METHODS FOR INTUBATION

(71) Applicant: The Board of Regents of the University of Texas System, Austin, TX (US)

(72) Inventor: Roberto Fernando Casal, Houston, TX (US)

(21) Appl. No.: 19/155,868

(22) PCT Filed: Feb. 14, 2024
(86) PCT No.: PCT/US2024/015723

§ 371 Date: Aug. 12, 2025

Related U.S. Application Data
(60) Provisional application No. 63/446,141, filed on Feb. 16, 2023.

(51) Int. Cl. A61B 1/00; A61B 1/267

(52) U.S. Cl. CPC A61B 1/0014; A61B 1/2676

(57) ABSTRACT

An apparatus includes a body extending along a first axis, the body including a first end and a second end opposite the first end, a first connector coupled with the body proximate the first end, the first connector defining a first opening configured to receive a portion of a working channel of a bronchoscope, a second connector coupled with the body proximate the second end and extending from the body at an angle relative to the first axis, the angle being greater than 0 degrees and less than 90 degrees. The second connector defines a second opening configured to receive a portion of an endotracheal tube while a portion of the bronchoscope is positioned within the endotracheal tube and a gap configured to allow the endotracheal tube to translate from outside the second opening to inside the second opening.

### CROSS-REFERENCE TO RELATED APPLICATIONS

The present application claims the benefit of and priority to U.S. Provisional Application No. 63/446,141, filed Feb. 16, 2023, the disclosure of which is incorporated herein by reference in its entirety.

### TECHNICAL FIELD

The present disclosure relates generally to subject intubation. More specifically, the present disclosure relates to systems and methods for bronchoscope endotracheal intubation.

### BACKGROUND

Subjects can be intubated for a number of reasons. Intubation can involve inserting an endotracheal tube (ETT) into a subject's trachea. Intubation procedures can be performed to facilitate breathing as well as to diagnosis conditions of the subject.

### SUMMARY

At least one aspect relates to an endotracheal clip apparatus for over-bronchoscope endotracheal intubation. The apparatus can have a first, upper end to be positioned around an entry port of a working channel of a bronchoscope, and a second, lower end to connect with a proximal end of an ETT, enabling the ETT to be coupled with the bronchoscope. The apparatus can be used as a sterilized, disposable, and/or single use device, and can have features to facilitate coupling of the apparatus, ETT, and/or bronchoscope, such as to facilitate the ease of and safety of performing over-bronchoscope endotracheal intubation procedures.

In some implementations, the apparatus includes a body extending along a first axis, the body including a first end and a second end opposite the first end, a first connector coupled with the body proximate the first end, the first connector defining a first opening to receive a portion of a working channel of a bronchoscope, a second connector coupled with the body proximate the second end and extending from the body at an acute angle relative to the first axis, such as an angle greater than 0 degrees and less than 90 degrees. The second connector defines a second opening to receive a portion of an endotracheal tube while a portion of the bronchoscope is positioned within the endotracheal tube and a gap separating a first leg of the second connector and a second leg of the second connector, the gap sized to allow the endotracheal tube to translate from outside the second opening to inside the second opening.

According to various implementations, the angle is between 120 degrees and 55 degrees. The angle can be between 80 degrees and 55 degrees. The angle can be between 95 degrees and 155 degrees. The first connector can define an enclosed circular first connector body. The second opening can define a first dimension and the gap can define a gap distance, the first dimension being greater than the gap distance. The second opening can be an oval opening that further defines a second dimension that can be smaller than the first dimension. A distance between the first end and the second end can define a body length between 10 cm and 18 cm. The first connector and the second connector can be integrally formed with the body. The body can be formed of a polymer material. The apparatus can include a slot (e.g., slot portion) positioned between the body and the second connector. The portion of the endotracheal tube that the second opening is to receive can be a first endotracheal tube portion, and the slot portion can define a third opening structured to receive a second portion of the endotracheal tube while the portion of the bronchoscope is positioned within the endotracheal tube. The slot portion can include a slot leg defining at least a portion of the third opening. The slot leg can inhibit rotation of the endotracheal tube relative to the portion of the bronchoscope positioned within the endotracheal tube while the second portion of the endotracheal tube is positioned within the third opening. The second portion of the endotracheal tube to be received by the third opening can be a wing extending from the endotracheal tube.

In some implementations, an apparatus includes a body extends from a first end to a second end, a first connector extending from the body proximate the first end along a first axis, the first connector defining a first opening to receive a portion of a bronchoscope, and a second connector extending from the body proximate the second end along a second axis, the second connector defining a second opening to receive a portion of an endotracheal tube while a portion of the bronchoscope can be positioned within the endotracheal tube, wherein the first axis and the second axis form an angle greater than 0 degrees.

The second connector can further define a gap separating a first leg of the second connector and a second leg of the second connector, the gap sized to allow the endotracheal tube to translate from outside the second opening to inside the second opening. The angle can be between 120 degrees and 90 degrees. The angle can be between 80 degrees and 55 degrees. The first connector can define an enclosed circular first connector body. The second opening can define a first dimension and the gap can define a gap distance, the first dimension being greater than the gap distance. The second opening can be an oval opening that further defines a second dimension that can be smaller than the first dimension. A distance between the first end and the second end can defines a body length between 10 cm and 18 cm. The first connector and the second connector can be integrally formed with the body. The apparatus can include a slot portion positioned between the body and the second connector and can define a third opening structured to receive a second portion of the endotracheal tube while the portion of the bronchoscope is positioned within the endotracheal tube. The slot portion can include a slot leg defining at least a portion of the third opening. The slot leg can inhibit rotation of the endotracheal tube relative to the portion of the bronchoscope positioned within the endotracheal tube while the second portion of the endotracheal tube is positioned within the third opening.

In some implementations, an apparatus for selectively coupling an endotracheal tube with a bronchoscope includes a body extending along a first axis, the body including a first end and a second end opposite the first end, a distance between the first end and the second end defining a length between 10 and 18 cm, a first connector coupled with the body proximate the first end, the first connector defining a first opening to receive a portion of a working channel of the bronchoscope, the first opening having a diameter between 1 cm and 3 cm, a second connector coupled with the body proximate the second end and extending from the body at an angle relative to the first axis, the angle being greater than 0 degrees and less than 90 degrees. The second connector can define a second opening to receive a portion of the endotracheal tube while a portion of the bronchoscope can be positioned within the endotracheal tube, the second opening defining a first dimension between 0.8 cm and 1.6 cm and a gap separating a first leg of the second connector and a second leg of the second connector, the gap defining a third dimension between 0.6 cm and 1 cm. The angle can be between 120 degrees and 55 degrees. The angle can be between 80 degrees and 55 degrees.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 is a perspective view of an endotracheal tube coupled with an bronchoscope via an apparatus, according to an example implementation;

FIG. 2 is a side view of the apparatus of FIG. 1;

FIG. 3 is a perspective view of the apparatus of FIG. 1;

FIG. 4 is a partial view of a first end of the apparatus of FIG. 1;

FIG. 5 is a partial view of a second end of the apparatus of FIG. 1;

FIG. 6 is a flow diagram for a method of performing an endotracheal intubation, according to an example implementation;

FIG. 7 is a perspective view of an apparatus, according to an example implementation;

FIG. 8 is a perspective view of an endotracheal tube coupled with an bronchoscope via the apparatus of FIG. 7;

FIG. 9 is a partial perspective view of a second end of the apparatus of FIG. 7 coupled with the endotracheal tube;

FIG. 10 is a partial perspective view of the second end of the apparatus of FIG. 7 coupled with the endotracheal tube;

FIG. 11 is a side view of the endotracheal tube oriented for intubation and coupled with the bronchoscope via the apparatus of FIG. 7; and

FIG. 12 is a side view of a second connector of the apparatus of FIG. 7 coupled and decoupled with the endotracheal tube.

### DETAILED DESCRIPTION

Below are detailed descriptions of various concepts related to, and implementations of, techniques, approaches, methods, apparatuses, and systems for subject intubation, such as to intubate a patient. The various concepts introduced above and discussed in greater detail below can be implemented in any of numerous ways, as the described concepts are not limited to any particular manner of implementation. Examples of specific implementations and applications are provided primarily for illustrative purposes.

Endotracheal tubes (ETT) are hollow tubes that can be used to intubate a subject. The endotracheal tube can be inserted through the mouth and into the trachea, to keep the airway open. For subjects with lung, heart, or chest problems, an endoscopic camera can be provided using a bronchoscope canto guide this insertion.

Using the flexible bronchoscope to guide the endotracheal tube can facilitate positioning the endotracheal tube in a target position. For example, if a subject has a compromised or otherwise difficult airway to navigate, the endotracheal tube is a double lumen tube, or the subject has an unstable cervical spine and requires intubation, using a flexible bronchoscope to guide the endotracheal tube can reduce risks involved with intubation of the subject, including by providing visual feedback for the operator performing the intubation.

The intubation can be performed as an over-bronchoscope procedure. For example, the ETT can be positioned proximate the bronchoscope. The ETT can be secured to the bronchoscope using tape or rubber bands, such as to prevent the ETT from sliding relative to the subject or bronchoscope, or from interfering with a hand of the operator that is manipulating a distal end of the bronchoscope. The distal end of the bronchoscope can be introduced through an upper airway of the subject, such as to be passed through the vocal cords and into a mid-trachea region. The tape or rubber band is released (e.g., quickly released) to allow the ETT to be advanced over the bronchoscope, such as until the ETT is visible in the trachea. The bronchoscope can be removed to leave the ETT in a target position.

To perform such procedures effectively, it can be useful for the ETT and bronchoscope to be connected in a sterile, easily releasable manner. For example, the subject can be paralyzed and/or not breathing, placing time constraints on how rapidly the ETT can be properly deployed at the target position. Where tape is used to secure the ETT with the bronchoscope, the tape can be difficult to remove, particularly where the operator is wearing gloves; a second operator can need to remove their gloves to remove the tape, or scissors can need to be used. Moreover, for the tape to properly connect the ETT with the bronchoscope, a substantial length of tape can be needed, which can increase the difficulty of removing the tape. Where non-medical devices such as rubber bands are used to secure the ETT with the bronchoscope, the rubbers cannot be sterile or readily available, and can stay around the ETT once released from the bronchoscope.

Referring to the figures generally, systems and methods for intubating a subject are described that can facilitate more rapid and sterile procedures, including by using apparatuses (e.g., endotracheal clips) as described herein that can be sterilized and can allow for more rapid release of the ETT from the bronchoscope at an appropriate time during the procedures. The intubation can involve an endotracheal tube, a bronchoscope, and an apparatus for selectively coupling the endotracheal tube to the bronchoscope. For example, the endotracheal intubation can be performed by guiding an endotracheal tube over a flexible bronchoscope.

According to various implementations, a system for performing an intubation can include an endotracheal tube, a bronchoscope, and an apparatus for selectively coupling the endotracheal tube to the bronchoscope. The apparatus includes a body extending along a first axis. The body including a first end and a second end opposite the first end. The apparatus includes a first connector coupled with the body proximate the first end. The first connector defines a first opening to receive a portion of a working channel of the bronchoscope. For example, the working channel of the bronchoscope can include a projection extending from a main body of the bronchoscope. The first connector can be sized to fit over the projection such that a portion of the projection is positioned within the first opening to couple the apparatus to the bronchoscope. In use, the bronchoscopy can be used in a generally vertical orientation such that the apparatus hangs from the working channel of the bronchoscope via the first connector.

The apparatus can include a second connector coupled with the body proximate the second end. The second connector defines a second opening configured to receive a portion of the endotracheal tube while a portion of the bronchoscope is positioned within the endotracheal tube. For example, the second opening can be used to couple the apparatus to the endotracheal tube while the first connector is coupled with the bronchoscope.

Various components of the apparatus, such as the body, first connector, and/or second connector, can be integrally or monolithically formed, or can be made of separate members that can be coupled with one another during manufacturing or prior to use.

The second connector can define a gap separating a first leg of the second connector and a second leg of the second connector, the gap sized or shaped to allow the endotracheal tube to translate from outside the second opening to inside the second opening.

According to various implementations, the endotracheal tube defines a shoulder proximate a first end of the endotracheal tube. In use, a portion of the bronchoscope is provided within the endotracheal tube and the endotracheal tube is brought towards the working channel of the bronchoscope such that the portion of the bronchoscope translates within the endotracheal tube. The apparatus can then be coupled with the working channel of the bronchoscope and the endotracheal tube such that the shoulder of the endotracheal tube interfaces with the second connector to limit translation of the endotracheal tube relative to the bronchoscope. As discussed below, the apparatus can include a second connector that compresses (e.g., applies force against) against the side of the endotracheal tube to facilitate preventing translation of the endotracheal tube relative to the bronchoscope.

According to various implementations, a portion of the bronchoscope extends out of a second end of the endotracheal tube. The bronchoscope can then be inserted into a desired location within the subject. Once the bronchoscope is in a desired location, the second connector can be decoupled from the endotracheal tube. The first connector can remain coupled with the working channel of the bronchoscope after the second connector is decoupled from the endotracheal tube. After the second connector is decoupled from the endotracheal tube, the second connector no longer limits translation of the endotracheal tube relative to the bronchoscope such that the endotracheal tube can be guided down the bronchoscope that is positioned within the endotracheal tube into a desired location.

According to various implementations, the second connector extends from the body at an angle relative to the first axis, such as an acute angle greater than 0 degrees and less than 90 degrees. Since the working channel is not axially aligned with the endotracheal tube, angling the second connector allows apparatus to simultaneously be coupled with the working channel and the endotracheal tube. According to various implementations, the angle is greater than 45 degrees. According to various implementations, the angle is between 80 degrees and 55 degrees.

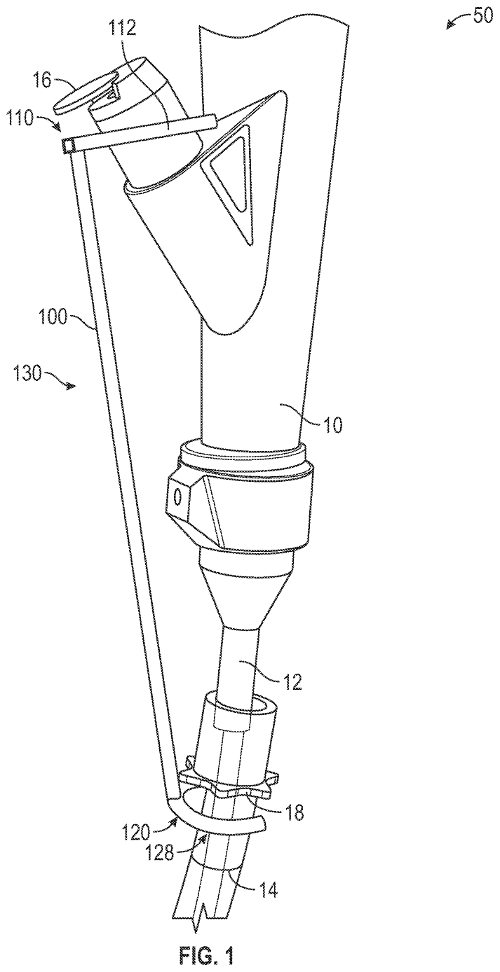

Referring now to FIG. 1, a perspective view of an intubation system 50 including an endotracheal tube 14 coupled with a bronchoscope 10 via an apparatus 100 is shown, according to an example implementation. As shown, the bronchoscope 10 includes a flexible portion 12 to be inserted into a subject (e.g., into the mouth, into the nose, etc.). For example, the bronchoscope 10 can include a camera proximate an end of the flexible portion 12 that enables an operator of the bronchoscope 10 to receive visual feedback while inserting the flexible portion 12 of the bronchoscope 10 into the subject.

According to various implementations, the bronchoscope 10 includes a working channel 16. As shown, the working channel 16 includes a projection that extends from the body of the bronchoscope 10. The working channel 16 can define an opening that is in communication with an opening defined by the flexible portion 12. According to various implementations, various instrumentation (e.g., a camera) can inserted into the working channel 16 and into an opening in the flexible portion 12.

As shown, the flexible portion 12 of the bronchoscope 10 is positioned within an endotracheal tube 14. The endotracheal tube 14 defines an inner opening that is large enough to receive the flexible portion 12 of the bronchoscope 10 such that endotracheal tube 14 can translate along the flexible portion 12 while the flexible portion 12 is within the endotracheal tube 14.

As is discussed further herein, the endotracheal tube 14 and the bronchoscope 10 can be utilized as a part of a bronchoscope endotracheal intubation process. As a part of the process, the endotracheal tube 14 receives the flexible portion 12 of the bronchoscope 10 (e.g., the flexible portion 12 is inserted into the endotracheal tube 14 as shown in FIG. 1; the endotracheal tube 14 receives and/or couples with the flexible portion 12). Once the endotracheal tube 14 receives the flexible portion 12 of the bronchoscope 10, the apparatus 100 can be utilized to selectively limit translation of the endotracheal tube 14 relative to the flexible portion 12. As shown, the apparatus 100 includes a body portion 132 that extends from a first end 110 to a second end 120. The apparatus 100 includes a first connector 112 proximate the first end 110 (e.g., a ring) and a second connector 128 (e.g., a clip) proximate the second end 120 of the apparatus 100. As shown, the first connector 112 is coupled with the projection of the working channel 16 and the second connector 128 is coupled with the endotracheal tube 14 to selectively limit translation of the endotracheal tube 14 relative to the flexible portion 12. For example, the endotracheal tube 14 can define a shoulder 18 that is configured to interface with the second connector 128 to prevent the shoulder 18 of the endotracheal tube 14 from translating past the second connector 128, which can thereby limit relative translation between the flexible portion 12 and the endotracheal tube 14.

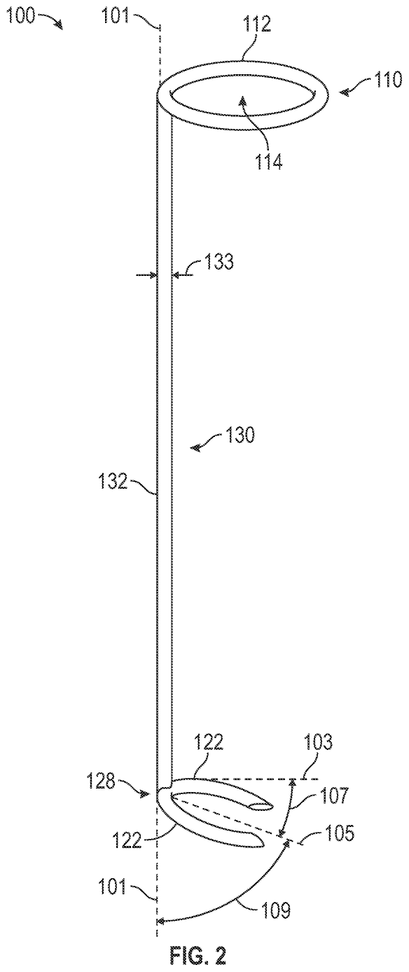

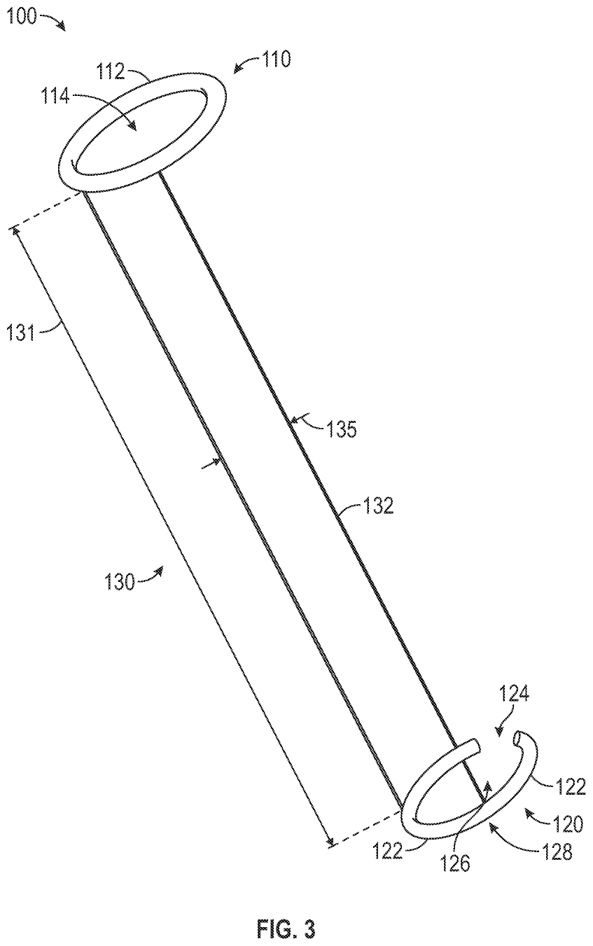

Referring now to FIGS. 2 and 3, a side view and a perspective view of the apparatus 100 are shown, respectively, according to an example implementation. As shown, the apparatus 100 includes a first connector 112 coupled with a body 130 proximate the first end 110. The first connector 112 defines an opening 114 (e.g., a first opening) configured to receive a projection of the working channel 16 to couple the apparatus 100 to the bronchoscope 10. For example, the bronchoscope 10 can be used in a relatively upright orientation (e.g., as shown in FIG. 1), such that the apparatus 100 hangs from the working channel 16 via the first connector 112.

As shown, the apparatus 100 can include a second connector 128 coupled with the body 130 proximate the second end 120. The second connector 128 is defined, for example, by at least one of a first leg 122 and a second leg 122 proximate the second end 120. The first leg 122 and the second leg 122 can define a gap 124 to allow the endotracheal tube 14 to be selectively received within an opening 126 (e.g., a second opening) defined by the second connector 128 to selectively couple the apparatus 100 to the endotracheal tube 14 (e.g., as shown in FIG. 1). For example, to define the gap 124, an end of the first leg 122 opposite from where the first leg 122 connects with the body portion 132 can be spaced from an end of the second leg 122 opposite from where the second leg 122 connects with the body portion 132. As discussed further below, the gap 124 can be smaller than an outer dimension of the endotracheal tube 14 such that the first leg 122 and the second leg 122 flex as the endotracheal tube 14 passes through the gap 124. In this sense, the first leg 122 and the second leg 122 can act as a spring to couple the second connector 128 of the apparatus 100 to the endotracheal tube 14. Therefore, a minimum threshold force can be required to overcome the spring force of the first leg 122 and the second leg 122 to move the endotracheal tube 14 from within the opening 126 to outside the opening 126 and vice versa.

The body 130 can include a body portion 132 that extends along a first axis 101 between the first end 110 and the second end 120. The distance between the first end 110 and the second end 120 defines a body length 131. According to various implementations, the body length 131 is between 5 cm and 25 cm. The body length 131 can be between 10 cm and 18 cm. The body length 131 can be between 12 cm and 16 cm. For example, the body length 131 can be approximately 14 cm.

The first connector 112 extends from the body portion 132 in a first direction. According to various implementations, the first connector 112 extends in a direction that is substantially perpendicular (e.g., +/−10%) to the first axis 101, which can facilitate alignment of the bronchoscope 10 with the endotracheal tube 14. For example, the first connector 112 can extend in a direction parallel to a second axis 103, wherein the second axis 103 is perpendicular to the first axis 101. In some implementations, the first connector 112 extends in a direction that is non-perpendicular to the first axis 101, e.g., less than 80 degrees or more than 100 degrees angled relative to the first axis 101.

The first leg 122 and the second leg 122 can extend along a third axis 105. The first axis 101 and the third axis 105 define a first angle 109. The first angle 109 can be between 120 degrees and 55 degrees. As shown, the first angle 109 is acute. The first angle 109 can be between 0 degrees and 90 degrees. For example, the first angle 109 can be greater than 45 degrees. For example, the first angle 109 can be between 80 degrees and 55 degree. As depicted in FIG. 1, by arranging the components of the apparatus 100 so that the first angle 109 is acute, the third axis 105 can be arranged to be substantially parallel to the shoulder 18 while the first connector 112 is coupled with the working channel 16, which can help prevent the shoulder 18 from translating beyond the second connector 128.

In some implementations, the first angle 109 may be a right angle or an obtuse angle. For example, the first angle 109 may be 90 degrees or greater. The first angle 109 can be between 120 degrees and 90 degrees. According to various embodiments, making the first angle 109 an obtuse angle may reduce the length of the apparatus 100, which may reducing packaging requirements, and/or can enable the apparatus 100 to have a compact form factor while deployed, which can facilitate performing the intubation and/or enable greater visibility for the operator during the intubation. The second connector 128 can elastically deform responsive to contacting the shoulder 18 (see FIG. 1) such that the third axis 105 is relatively parallel to the shoulder 18 when under an axial load, which can help prevent the shoulder 18 from translating beyond the second connector 128.

In various implementations in which the first angle 109 is between 0 and 90 degrees, the second axis 103 and the third axis 105 can define a second angle 107 between 0 degrees and 90 degrees. The second angle 107 can be less than 45 degrees. For example, the second angle 107 can be between 10 degrees and 35 degrees. According to various implementations, the first connector 112 is parallel to the second axis 103 such that the second angle 107 defines the relative angle between the first connector 112 and the second connector 128, which is defined by the first leg 122 and the second leg 122.

According to various implementations, providing the apparatus 100 with the first connector 112 and the second connector 128 that are angled relative to one another (e.g., where the angle between the first connector 112 and the body portion 132 is different than the angle between the second connector 128 and the body portion 132) and coupled with one another (e.g., via the body portion 132) can facilitate coupling the second connector 128 to the endotracheal tube 14 while the first connector 112 is coupled with the working channel 16 of the bronchoscope 10. For example, the working channel 16 of the bronchoscope 10 can be axially offset from the flexible portion 12 of the bronchoscope 10 (e.g., as shown in FIG. 1). Thus, the relative angle between the first connector 112 and the second connector 128 allows an operator of the apparatus 100 to selectively couple the second connector 128 to the endotracheal tube 14 that receives the flexible portion 12 of the bronchoscope 10.

According to various implementations, the first connector 112 is integrally formed with the body portion 132. The second connector 128 can be integrally formed with the body portion 132. Integral formation of the first connector 112 and/or second connector 128 with the body portion 132 can allow for a more compact and/or structurally resilient device. According to various implementations, the apparatus 100 is formed from a single piece of material. For example, the apparatus 100 can be formed from a polymer material. The polymer material can be provide some flexibility in the apparatus 100. For example, as described above, the first leg 122 and the second leg 122 can flex as the endotracheal tube 14 is inserted through the gap 124. The apparatus 100 can be formed of a material that can be easily sterilized. For example, the apparatus 100 can be relatively heat resistant and/or corrosion resistant. The apparatus 100 can be a disposable product, which can reduce the risk of undesired contamination. Further, the apparatus 100 can be sterilized and transported with packaging such that the apparatus 100 is sterile when removed from the packaging. In some implementations, the apparatus 100 can include one or more removable components, such as to allow for one or more portions of the apparatus 100 to be reused and/or replaced with components of varied sizing to account for various sizes of bronchoscopes, ETTs, and/or subject anatomies.

The body portion 132 can be rigid such that the body length 131 is relatively fixed (e.g., have a greater rigidity than a threshold rigidity to prevent deformation more than a target percentage, e.g. five percent, under an expected load, force, and/or torque during usage). In some implementations, the body portion 132 includes a material that can elastically deform under certain loads such that the body length 131 varies under different loads. The body portion 132 further defines a width 135. The width 135 can be between 0.5 cm and 2.5 cm. For example, the width 135 can be 1.5 cm.

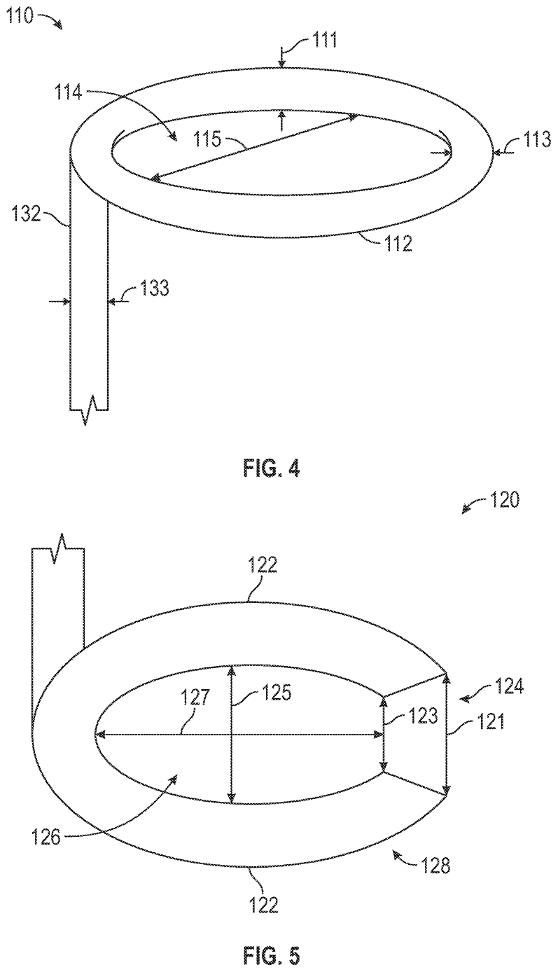

Referring now to FIG. 4, a partial view of the first end 110 of the apparatus 100 is shown, according to an example implementation. As shown, the body portion 132 defines a width 133. The width 133 can be between 0.5 cm and 2.5 cm. For example, the width 133 can be 1.5 cm.

As shown, the first connector 112 defines a width 113 (e.g., the distance between an inner diameter (e.g., the dimension 115) of the opening 114 and the outer diameter of the first connector 112). The width 113 can be between 0.2 cm and 0.8 cm. For example, the width 113 can be 0.5 cm. The first connector 112 further defines a thickness 111 (e.g., a distance between an upper surface of the first connector 112 and a lower surface of the first connector 112). The thickness 111 can be between 0.1 cm and 0.3 cm. For example, the thickness 111 can be 0.2 cm. It should be appreciated that the second connector 128 can define a similar width and/or thickness.

As shown, the opening 114 defines a dimension 115 (e.g., a diameter). The dimension 115 is large enough to receive the working channel 16 of the bronchoscope 10, as is discussed further herein. The dimension 115 can be between 1.5 cm and 2.5 cm. For example, the dimension 115 can be 2 cm. As shown, the opening 114 is generally circular, however, the shape of the opening 114 can be altered depending on the shape of the working channel 16 that the apparatus 100 is coupled with.

Referring now to FIG. 5, a partial view of the second end 120 of the apparatus 100 is shown, according to an example implementation. As shown, the first leg 122 and the second leg 122 define a gap 124 configured to receive a portion of the endotracheal tube 14. As shown, the gap 124 defines a first dimension 123 (e.g., a gap distance) and a second dimension 121 (e.g., a gap distance). According to various implementations, the first dimension 123 is smaller than the second dimension 121. This arrangement can facilitate guiding the endotracheal tube 14 into the opening 126. For example, the larger second dimension 121 can be easier to guide an endotracheal tube 14 into, while the smaller first dimension 123 secure the endotracheal tube 14 within the opening 126 to reduce the likelihood of accidental decoupling of the second connector 128 to the endotracheal tube 14. The first dimension 123 can be smaller than an outer dimension of the endotracheal tube 14. According to various implementations, the first dimension 123 is between 0.5 cm and 1.1 cm. For example, the first dimension 123 can be 0.8 cm. According to various implementations, the second dimension 121 is between 0.8 cm and 1.3 cm. For example, the second dimension 121 can be 1 cm.

As shown, the opening 126 defines a first dimension 125 (e.g., minor axis) and a second dimension 127 (e.g., a major axis). As shown, the first dimension 125 is smaller than the second dimension 127 such that the opening 126 is oval shaped. The first dimension 125 of the opening 126 can be greater than the first dimension 123 of the gap 124. According to various implementations, the first dimension 125 is smaller than an outer dimension of the endotracheal tube 14. As such, when the endotracheal tube 14 is positioned within the opening 126, the first leg 122 and the second leg 122 apply a force to the outside of the endotracheal tube 14 to secure the apparatus 100 to the endotracheal tube 14. According to various implementations, the first dimension 125 is between 1 cm and 1.5 cm. For example, the first dimension 125 can be 1.2 cm. The second dimension 127 can be between 1.4 cm and 2.2 cm. For example, the second dimension 127 can be 1.8 cm. The first dimension 125 and the second dimension 127 can be equal.

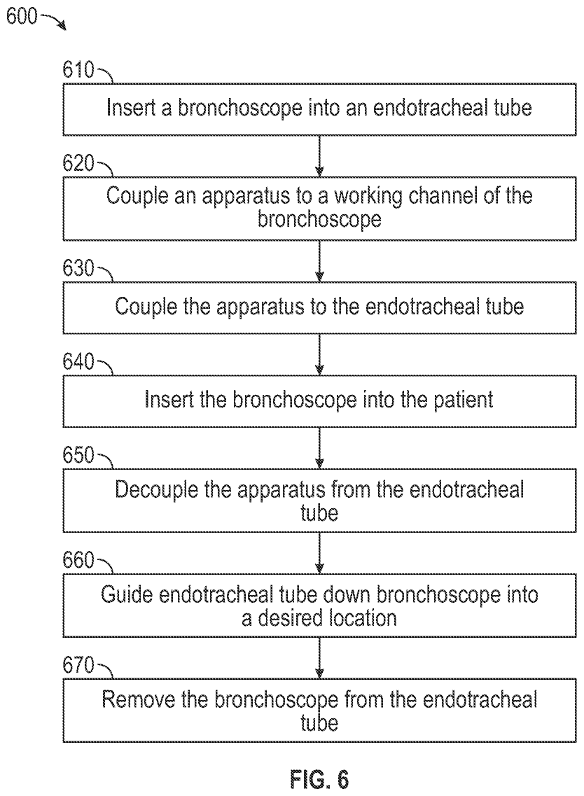

Referring now to FIG. 6, a flow diagram for a method 600 of performing an endotracheal intubation is shown, according to an example implementation. The method 600 can be performed using one or more items of the equipment described herein (e.g., the apparatus 100, the apparatus 700, the bronchoscope 10, the endotracheal tube 14, etc.). It should be appreciated that the method 600 need not be performed in the order shown. Further, various processes can be omitted and additional processes can be included.

The method 600 can be utilized for endotracheal intubation over a flexible bronchoscope. The method 600 can be utilized to intubate difficult airways, for insertion of double lumen tubes, and/or for subjects with and unstable cervical spine that require intubation.

At process 610, an endotracheal tube is received by a bronchoscope. For example, a flexible portion of the bronchoscope can be inserted into the endotracheal tube. According to various implementations, an end of the flexible portion (e.g., the end opposite a working channel of the bronchoscope) extends out of the end of the endotracheal tube.

At process 620, an apparatus (e.g., the apparatus 100, the apparatus 700) is coupled with a working channel of the bronchoscope. For example, a first connector (e.g., the first connector 112, the first connector 708) can be coupled with the working channel of the bronchoscope such that a portion of the working channel is positioned within an opening defined by the first connector. According to various implementations, the bronchoscope can subsequently be utilized in an upright position (e.g., as shown in FIG. 1) such that the apparatus hangs from the working channel via the first connector.

At process 630, the apparatus is coupled with the endotracheal tube. For example, a second connector (e.g., the second connector 128, the second connector 128) can receive a portion of the endotracheal tube while the flexible portion of the bronchoscope is positioned within endotracheal tube. The clamping forces of the first leg and the second leg of the second connector and/or the interaction between the second connector and a shoulder of the endotracheal tube can help prevent or otherwise reduce translation of the endotracheal tube along the flexible portion of the bronchoscope while the apparatus is coupled with the working channel and the endotracheal tube.

At process 640, the bronchoscope is inserted into the subject. For example, the flexible portion can be inserted into the mouth or nose of the subject and guided into a desired location in the trachea using visual feedback provided by a camera within the flexible portion of the bronchoscopes. While the flexible portion of the bronchoscope is inserted into the subject, the apparatus prevents the endotracheal tube from undesirably sliding down the flexible portion until the bronchoscope is in a desired location.

At process 650, the apparatus is decoupled from the endotracheal tube. For example, once the bronchoscope is in a desired location, the second connector can be decoupled from the endotracheal tube such that the apparatus no longer restricts relative movement between the endotracheal tube and the flexible portion of the bronchoscope. As described above, the second connector includes a gap that allows the second connector to clip onto and off of the endotracheal tube as desired.

At process 660, the endotracheal tube is guided down the bronchoscope and into a desired location. Once the apparatus is decoupled from the endotracheal tube, translation of the endotracheal tube along the flexible portion of the bronchoscope is no longer restricted. As such, the endotracheal tube can be safely guided down the bronchoscope into a desired location to intubate the subject.

At process 670, the bronchoscope is removed from the endotracheal tube, leaving an unobstructed air flow path through the endotracheal tube. The apparatus first connector can then be decoupled from the working channel and the apparatus can be disposed of and/or sterilized.

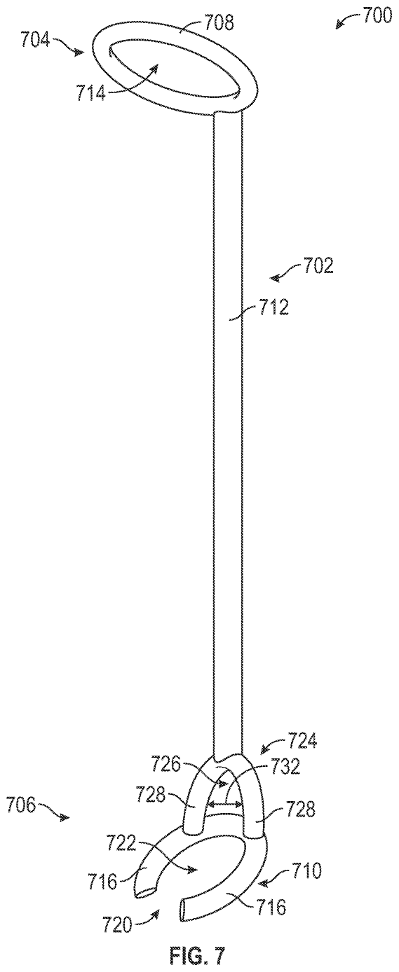

Referring to FIG. 7, a clip (e.g., an endotracheal clip) is shown as apparatus 700. The apparatus 700 can incorporate features of, be substantially similar to, the same as, and/or perform similar functions as the apparatus 100. The apparatus 700 can be utilized to selectively limit translation of the endotracheal tube 14 relative to the flexible portion 12.

As shown, the apparatus 700 includes a body 702 that extends from a first end 704 to a second end 706. The apparatus 700 includes a first connector 708 proximate the first end 704 (e.g., a ring, such as a closed loop structure) and a second connector 710 (e.g., a clip) proximate the second end 706 of the apparatus 700.

The apparatus 700 includes the first connector 708 coupled with a body portion 712 proximate the first end 704. The first connector 708 defines an opening 714 configured to receive a projection of the working channel 16 to couple the apparatus 700 to the bronchoscope 10. For example, the bronchoscope 10 can be used in a relatively upright orientation (e.g., as shown in FIG. 1), such that the apparatus 700 hangs from the working channel 16 via the first connector 708.

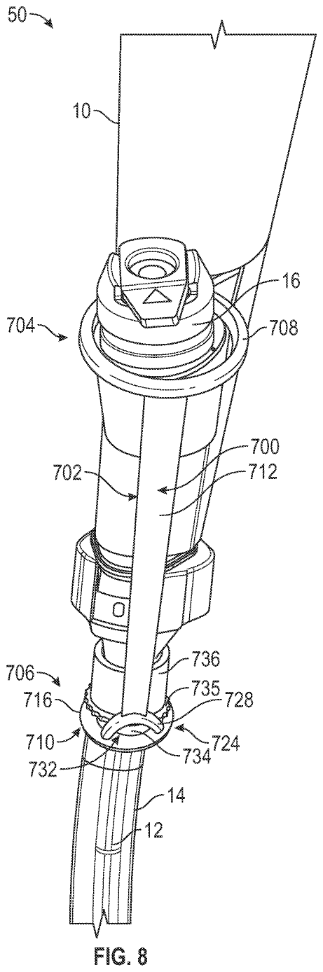

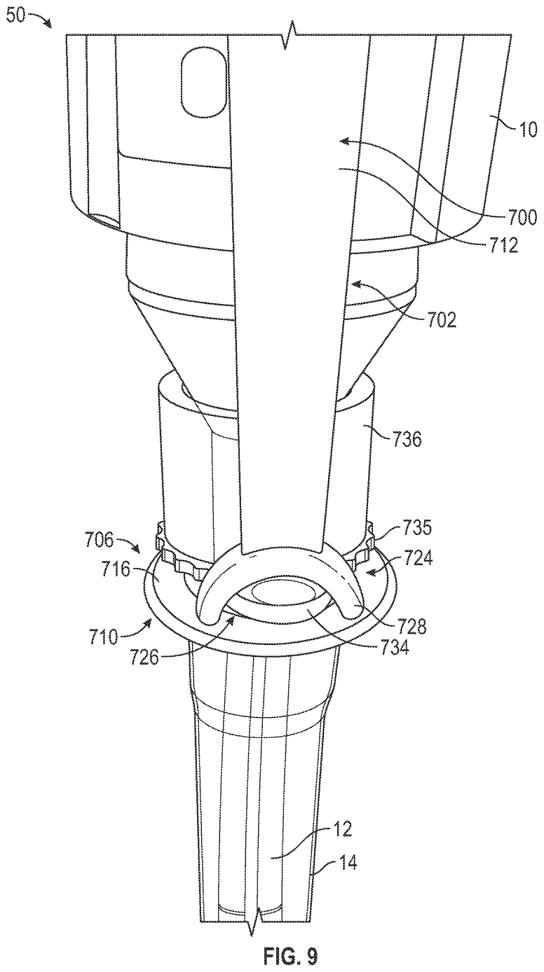

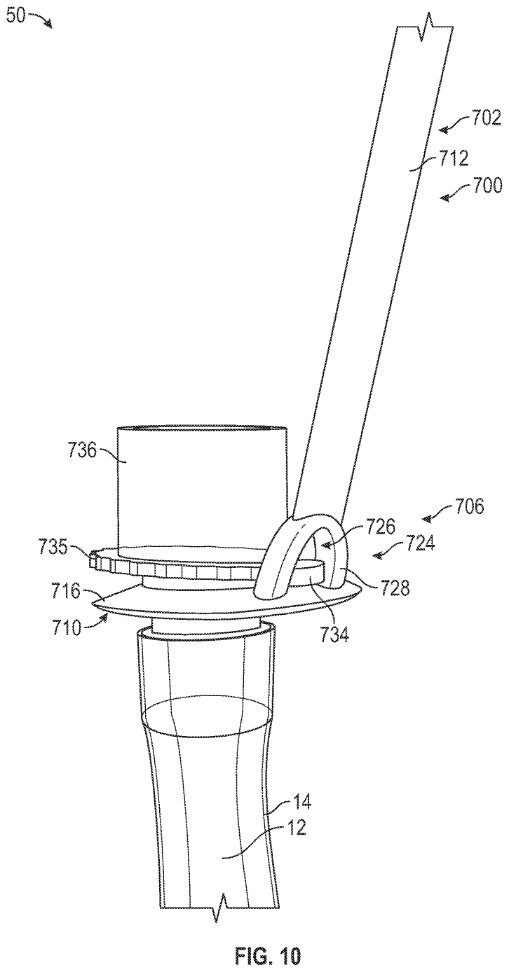

As shown, the apparatus 700 further includes the second connector 710 coupled with the body portion 712 proximate the second end 706. The second connector 710 is defined by a first leg 716 and a second leg 716 proximate the second end 706. The first leg 716 and the second leg 716 define a gap 720 that is configured to allow the endotracheal tube 14 to be selectively received within an opening 722 defined by the second connector 710 to selectively couple the apparatus 700 to the endotracheal tube 14 (e.g., as shown in FIGS. 8-10). The gap 720 can be structured and dimensioned substantially similar to the gap 124 as discussed in greater detail above with reference to FIGS. 2-5. For example, the gap 720 can be smaller than an outer dimension of the endotracheal tube 14 such that the first leg 716 and the second leg 716 flex as the endotracheal tube 14 passes through the gap 720. In this sense, the first leg 716 and the second leg 716 can act as a spring to couple the second connector 710 of the apparatus 700 to the endotracheal tube 14. Therefore, a minimum threshold force can be required to overcome the spring force of the first leg 716 and the second leg 716 to move the endotracheal tube 14 from within the opening 722 to outside the opening 722 and vice versa.

As shown, the apparatus 700 includes a slot (e.g., an eye-let), shown as slot portion 724, positioned proximate the second end 706 between the body portion 712 and the second connector 710. The slot portion 724 connects the body portion 712 to the second connector 710. The slot portion 724 defines an opening 726 (e.g., a third opening) shaped to receive at least a portion of the endotracheal tube 14 to inhibit translation of the endotracheal tube 14 relative to the flexible portion 12. The slot portion 724 includes a first leg 728 and a second leg 728 (e.g., a slot leg). The opening 726 can be defined by the first leg 728, the second leg 728, and at least a portion of the second connector 710 (e.g., at least a portion of the first leg 716, at least a portion of the second leg 716).

As shown, the opening 726 defines a dimension 732. The dimension 732 can be a distance between the first leg 728 and the second leg 728. For example, the dimension 732 can be a distance extending in a lateral direction between a portion of the first leg 728 facing the opening 726 and a portion of the second leg 728 facing the opening 726. The dimension 732 is large enough such that the opening 726 can receive at least a portion of the endotracheal tube 14 to inhibit translation of the endotracheal tube 14 relative to the flexible portion 12.

As shown in FIGS. 8-10, the first connector 708 is coupled with the projection of the working channel 16 and the second connector 710 is coupled with the endotracheal tube 14 to selectively limit translation of the endotracheal tube 14 relative to the flexible portion 12. For example, the endotracheal tube 14 can define the shoulder 18, and the first leg 716 and the second leg 716 of the second connector 710 can engage with (e.g., contact, interface with, etc.) the shoulder 18 to prevent the shoulder 18 of the endotracheal tube 14 from translating past the second connector 710, thereby limiting relative translation between the flexible portion 12 and the endotracheal tube 14.

As shown, an adapter 736 may facilitate inserting the flexible portion 12 of the bronchoscope 10 within the endotracheal tube 14 such that the endotracheal tube 14 can translate along the flexible portion 12 while the flexible portion 12 is within the endotracheal tube 14. The adapter 736 can be received by endotracheal tube 14 and extend out of the endotracheal tube 14 (e.g., the adapter 736 can include a first portion sized to be received in the endotracheal tube 14 and a second portion sized to be positioned outside of the endotracheal tube 14 while the first portion is received in the endotracheal tube 14). The portion of the adapter 736 received by the endotracheal tube 14 can define a diameter that is larger than the inner opening (e.g., inner diameter) of the endotracheal tube 14 such that when the adapter 736 is inserted into the endotracheal tube 14, a seal is created between the adapter 736 and the endotracheal tube 14. When the adapter 736 is inserted into the endotracheal tube 14, rotation of the adapter 736 relative to the endotracheal tube 14 can be restricted. For example, a friction force between the adapter 736 and the endotracheal tube 14 and/or a compressive force from the endotracheal tube 14 acting on the adapter 736 (e.g., due to an outer diameter of the adapter 736 being larger than an inner diameter of the endotracheal tube 14) can restrict rotation of the adapter 736 relative to the endotracheal tube 14. The adapter 736 can include an opening configured to receive the flexible portion 12 and facilitate inserting the flexible portion 12 into the endotracheal tube 14.

The adapter 736 can be structured to restrict rotation between the endotracheal tube 14 and the flexible portion 12 of the bronchoscope 10. The adapter 736 can include a shoulder 735 to engage with (e.g., contact, interface with, etc.) the first leg 716 and the second leg 716 of the second connector 710 to prevent the shoulder 735 of the adapter 736 from translating past the second connector 710, thereby limiting relative translation between the flexible portion 12 and the endotracheal tube 14.

The adapter 736 can include a wing 734 (e.g., a lip) that the first leg 716 and the second leg 716 of the second connector 710 can connect with to prevent the shoulder 735 (and the wing 734) from translating past the second connector 710. The wing 734 can extend outward from the endotracheal tube 14 in a direction that is substantially perpendicular to the endotracheal tube 14.

As shown, the wing 734 can be received within the opening 726 of the slot portion 724. The dimension 732 defined by the opening 726 is large enough to receive at least a portion of the wing 734. For example, the dimension 732 may be at least as large as a width of the wing 734. When the wing 734 is received within the opening 726 of the slot portion 724, the wing 734 can interface with the first leg 728 and the second leg 728 of the slot portion 724 to prevent the wing 734 from inadvertently or unintentionally being removed from within the opening 726, thereby limiting relative rotation between the endotracheal tube 14 and the flexible portion 12 of the bronchoscope 10.

The portion of the adapter 736 extending out of the endotracheal tube 14 can include the shoulder 735 and the wing 734. The adapter 736 can be received within the opening 722 defined by the second connector 710 to selectively couple the apparatus 700 to the endotracheal tube 14. For example, the shoulder 735 can interface with the first leg 716 and the second leg 716 of the second connector 710 to prevent the shoulder 735 of the adapter 736 from translating past the second connector 710, thereby limiting relative translation between the flexible portion 12 and the endotracheal tube 14. Similarly, the wing 734 can interface with the first leg 728 and the second leg 728 of the slot portion 724 to prevent the wing 734 and the adapter 736 from inadvertently or unintentionally being removed from within the opening 726, thereby limiting relative rotation between the endotracheal tube 14 and the flexible portion 12 of the bronchoscope 10. In some examples, the adapter 736 can be integrally formed with the endotracheal tube 14 such that the endotracheal tube 14 includes the shoulder 735 (e.g., the shoulder 18) and the wing 734.

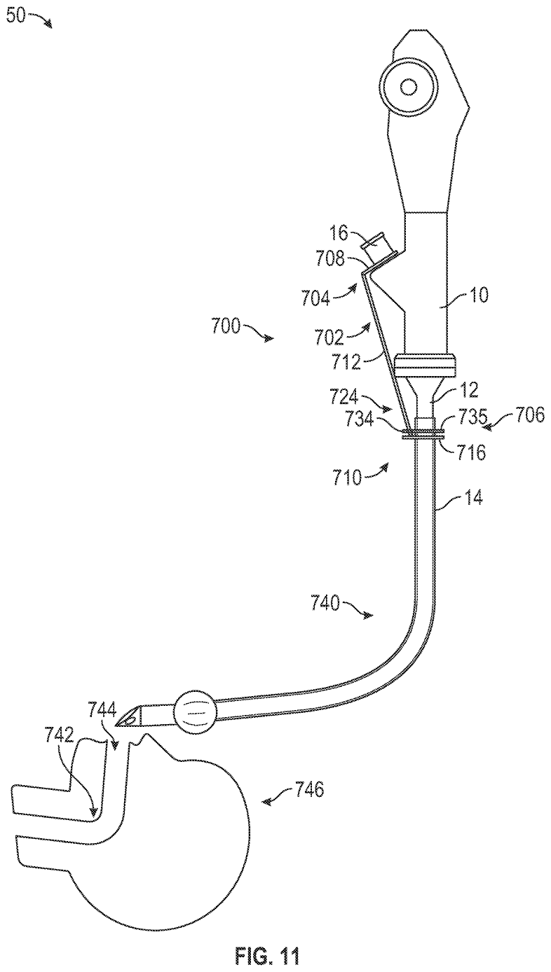

As shown in FIG. 11, the endotracheal tube 14 can define a curvature 740 and/or be capable (e.g., sufficiently flexible and/or resilient) to be bent into the curvature 740. During an intubation process when the bronchoscope 10 is inserted into a subject 746 (e.g., during the method 600 at process 640), it can be desired to orient the endotracheal tube 14 such that curvature 740 thereof is substantially aligned or matched with a curvature 742 of an anatomy 744 (e.g., an airway, a trachea, etc.) of the subject 746. For example, substantially aligning or matching the curvature 740 of the endotracheal tube 14 with the curvature 742 of the anatomy 744 of the subject 746 may include orienting the endotracheal tube 14 relative to the subject 746 such that a concavity of the curvature 740 is facing the same direction of a concavity of the anatomy 744 of the subject 746. Aligning or otherwise matching the curvature 740 of the endotracheal tube 14 with the curvature 742 of the anatomy 744 of the subject 746 can make inserting the bronchoscope 10 and the endotracheal tube 14 into the subject 746 easier.

The wing 734 can be oriented relative to the curvature 740 of the endotracheal tube 14 such that when the apparatus 700 is installed (e.g., when the apparatus 700 is coupled with the working channel 16 of the bronchoscope 10 and coupled with the endotracheal tube 14) and the wing 734 extends through the slot portion 724, the curvature 740 of the endotracheal tube 14 is substantially aligned with the curvature 742 of the anatomy 744 of the subject 746. The wing 734 can contact the first leg 728 and/or the second leg 728 of the slot portion 724 to inhibit relative rotation between the endotracheal tube 14 and the flexible portion 12 of the bronchoscope 10 such that, during the intubation process, the curvature 740 of the endotracheal tube 14 remains substantially aligned with the curvature 742 of the anatomy 744 of the subject 746. In such examples, an operator (e.g., a surgeon, a doctor, a nurse, etc.) does not have to rotate or orient (e.g., re-orient) the endotracheal tube 14 to align the curvature 740 with the curvature 742.

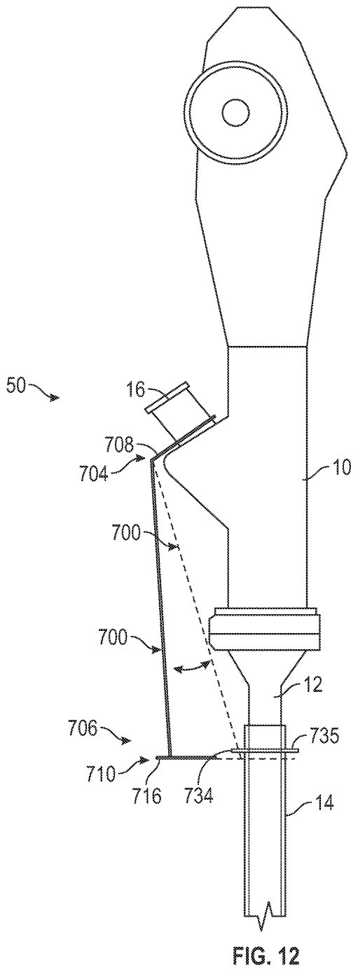

As shown in FIG. 12, the apparatus 700 can be coupled and decoupled from the endotracheal tube 14 and the working channel 16 of the bronchoscope 10. For example, the second connector 710 can receive a portion of the endotracheal tube 14 while the flexible portion 12 of the bronchoscope 10 is positioned within endotracheal tube 14. The clamping forces of the first leg 716 and the second leg 716 of the second connector 710, the interaction between the second connector 710 and the shoulder 735 (or the shoulder 18), and/or the reception of the wing 734 within the opening 726 of the slot portion 724 can help prevent or otherwise reduce (i) translation of the endotracheal tube 14 along the flexible portion 12 of the bronchoscope 10 and (ii) rotation of the endotracheal tube 14 relative to the bronchoscope 10 while the apparatus 700 is coupled with the working channel 16 and the endotracheal tube 14. By way of another example, once the bronchoscope 10 is in a desired location, the second connector 710 can be decoupled from the endotracheal tube 14 such that the apparatus 700 no longer restricts relative movement between the endotracheal tube 14 and the flexible portion 12 of the bronchoscope 10. In such examples, the second end 706 can pivot in a direction towards or away from the endotracheal tube 14 while the first connector 708 is coupled with the working channel 16. As described above, the second connector 710 includes the gap 720 that facilitates clipping the second connector 710 onto and off of the endotracheal tube 14 as desired.

While this specification contains many specific implementation details, these should not be construed as limitations on the scope of any inventions or of what can be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

Having now described some illustrative implementations and implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. Although the examples provided can be useful transforming a three-dimensional point cloud to a different reference frame, the systems and methods described herein can be applied to other environments. The foregoing implementations are illustrative rather than limiting of the described systems and methods. The scope of the systems and methods described herein can thus be indicated by the appended claims, rather than the foregoing description, and changes that come within the meaning and range of equivalency of the claims are embraced therein.

What is claimed is:

1. An endotracheal clip apparatus, comprising:
a body extending along a first axis, the body including a first end and a second end opposite the first end;
a first connector coupled with the body proximate the first end, the first connector defining a first opening to receive a portion of a working channel of a bronchoscope;
a second connector coupled with the body proximate the second end and extending from the body at an acute angle relative to the first axis, the second connector defining:
a second opening to receive a portion of an endotracheal tube while a portion of the bronchoscope is positioned within the endotracheal tube; and
a gap separating a first leg of the second connector and a second leg of the second connector, the gap shaped to allow the endotracheal tube to translate from outside the second opening to inside the second opening.

2. The endotracheal clip apparatus of claim 1, wherein the acute angle is greater than 45 degrees.

3. The endotracheal clip apparatus of claim 1, wherein the acute angle is between 80 degrees and 55 degrees.

4. The endotracheal clip apparatus of claim 1, wherein the first connector defines an enclosed circular first connector body.

5. The endotracheal clip apparatus of claim 1, wherein the second opening defines a first dimension and the gap defines a gap distance, the first dimension being greater than the gap distance.

6. The endotracheal clip apparatus of claim 5, wherein the second opening is an oval opening that further defines a second dimension that is greater than the first dimension.

7. The endotracheal clip apparatus of claim 1, wherein a distance between the first end and the second end defines a body length between 10 cm and 18 cm.

8. The endotracheal clip apparatus of claim 1, wherein the first connector and the second connector are integrally formed with the body.

9. The endotracheal clip apparatus of claim 1, further comprising a slot portion positioned between the body and the second connector.

10. The endotracheal clip apparatus of claim 9, wherein the portion of the endotracheal tube that the second opening is shaped to receive is a first endotracheal tube portion, and wherein the slot portion defines a third opening to receive a second portion of the endotracheal tube while the portion of the bronchoscope is positioned within the endotracheal tube.

11. The endotracheal clip apparatus of claim 10, wherein the slot portion incudes a slot leg defining at least a portion of the third opening, and wherein the slot leg is to restrict rotation of the endotracheal tube relative to the portion of the bronchoscope positioned within the endotracheal tube while the second portion of the endotracheal tube is positioned within the third opening.

12. The endotracheal clip apparatus of claim 11, wherein the second portion of the endotracheal tube to be received by the third opening is a wing extending from the endotracheal tube.

13. An apparatus, comprising:
a body extending from a first end to a second end;
a first connector extending from the body proximate the first end along a first axis, the first connector defining a first opening to receive a portion of a bronchoscope; and
a second connector extending from the body proximate the second end along a second axis, the second connector defining a second opening to receive a portion of an endotracheal tube while a portion of the bronchoscope is positioned within the endotracheal tube, wherein the first axis and the second axis form an angle between 45 degrees and 120 degrees.

14. The apparatus of claim 13, wherein the second connector further defines a gap separating a first leg of the second connector and a second leg of the second connector, the gap shaped to allow the endotracheal tube to translate from outside the second opening to inside the second opening.

15. The apparatus of claim 13, wherein the angle is between 120 degrees and 90 degrees.

16. The apparatus of claim 13, wherein the angle is between 80 degrees and 55 degrees.

17. The apparatus of claim 13, further comprising a slot portion positioned between the body and the second connector and defining a third opening structured to receive a second portion of the endotracheal tube while the portion of the bronchoscope is positioned within the endotracheal tube.

18. The apparatus of claim 17, wherein the slot portion incudes a slot leg defining at least a portion of the third opening, and wherein the slot leg is to restrict rotation of the endotracheal tube relative to the portion of the bronchoscope positioned within the endotracheal tube while the second portion of the endotracheal tube is positioned within the third opening.

19. An endotracheal clip apparatus for selectively coupling an endotracheal tube to a bronchoscope, the endotracheal clip apparatus comprising:
a body extending along a first axis, the body including a first end and a second end opposite the first end, a distance between the first end and the second end defining a length between 10 and 18 cm;
a first connector coupled with the body proximate the first end, the first connector defining a first opening to receive a portion of a working channel of the bronchoscope, the first opening having a diameter between 1 cm and 3 cm;
a second connector coupled with the body proximate the second end and extending from the body at an angle relative to the first axis, the angle being greater than 0 degrees and less than 120 degrees, the second connector defining:
a second opening to receive a portion of the endotracheal tube while a portion of the bronchoscope is positioned within the endotracheal tube, the second opening defining a first dimension between 0.8 cm and 1.6 cm; and
a gap separating a first leg of the second connector and a second leg of the second connector, the gap defining a third dimension between 0.6 cm and 1 cm.

20. The endotracheal clip apparatus of claim 19, wherein the angle is between 120 degrees and 55 degrees.
